(19) United States
(12) Patent Application Publication
(10) Pub. No.: US 2013/0144328 A1
(43) Pub. Date: Jun. 6, 2013

(54) EXPANDING DISTAL SHEATH WITH COMBINED EMBOLIC PROTECTION

(71) Applicant: Boston Scientific Scimed, Inc., Maple Grove, MN (US)

(72) Inventors: Jan Weber, Maastricht (NL); James M. Anderson, Fridley, MN (US)

(73) Assignee: BOSTON SCIENTIFIC SCIMED, INC., Maple Grove, MN (US)

(21) Appl. No.: 13/688,292

(22) Filed: Nov. 29, 2012

Related U.S. Application Data
(60) Provisional application No. 61/567,264, filed on Dec. 6, 2011.

(51) Int. Cl. A61F 2/958

(52) U.S. Cl. CPC A61F 2/958
USPC 606/200

(57) ABSTRACT

A medical device may include an elongate shaft having a distal end portion and a balloon disposed at least partially within the distal end portion. The distal end portion may be configured to selectively expand from a collapsed delivery configuration to a distally-opening expanded filtering configuration. A method of deploying a medical device may include obtaining an elongate shaft having a distal end portion and a balloon disposed at least partially within the distal end portion, advancing the elongate shaft through a vessel to a treatment site, disposing at least a portion of the balloon within the treatment site with the distal end of the elongate shaft positioned adjacent the treatment site, at least partially inflating the balloon, thereby expanding the distal end portion, and deflating the balloon and subsequently performing a procedure with the distal end of the elongate shaft positioned within the vessel adjacent to the treatment site.

### TECHNICAL FIELD

The invention relates generally to medical devices and more particularly to medical devices that are adapted for use in repairing heart valves.

### BACKGROUND

Aortic valve stenosis is a frequent expression of valvular heart disease, and may often be a leading indicator for balloon valvuloplasty and/or valve replacement therapy in

Europe and the United States. The prevalence of aortic stenosis tends to increase in older population groups. In some cases, balloon valvuloplasty and/or valve replacement therapy may loosen and release vulnerable plaque affecting the aortic valve as well as emboli caused or released by the procedure itself This debris may travel downstream where it may lodge in and/or obstruct a smaller vessel, presenting additional risk to the patient.

A continuing need exists for improved distal protection devices and methods for use in conjunction with balloon valvuloplasty and/or valve replacement surgery.

### SUMMARY

A medical device may include an elongate shaft having a proximal end, a distal end portion including a distal end, and a balloon disposed at least partially within the distal end portion. The distal end portion may be configured to selectively expand from a collapsed delivery configuration to a distally-opening expanded filtering configuration. The distal end may have a first outer diameter in the collapsed delivery configuration and a second outer diameter in the expanded filtering configuration.

A method of deploying a medical device at a treatment site may include obtaining an elongate shaft having a proximal end, a distal end portion including a distal end, and a balloon disposed at least partially within the distal end portion and extending distally therefrom, wherein the distal end portion is configured to selectively expand from a collapsed delivery configuration to a distally-opening expanded filtering configuration, advancing the elongate shaft through a vessel in a retrograde direction to the treatment site, disposing at least a portion of the balloon within the treatment site such that the distal end of the elongate shaft is positioned adjacent the treatment site, at least partially inflating the balloon, thereby expanding the distal end of the elongate shaft to the expanded filtering configuration, wherein the distal end substantially conforms to an inner surface of the vessel adjacent to the treatment site, and deflating the balloon and subsequently performing a valvectomy, valvuloplasty, or transcatheter aortic valve implantation procedure with the distal end of the elongate shaft positioned within the vessel adjacent to the treatment site in the expanded filtering configuration.

Although discussed with specific reference to use within the coronary vasculature of a patient, for example to repair a heart valve, embolic protection devices and methods of use in accordance with the disclosure can be adapted and configured for use in other parts of the anatomy, such as the digestive system, the respiratory system, or other parts of the anatomy of a patient.

### BRIEF DESCRIPTION OF THE FIGURES

FIG. 1 is a partial side view of a distal end of an elongate shaft of an example medical device in a collapsed delivery configuration;

FIG. 1A is a partial side view of the distal end of the elongate shaft of the example medical device of FIG. 1 in an expanded filtering configuration;

FIG. 2 is a partial side view of a distal end of an elongate shaft of an example medical device in a collapsed delivery configuration;

FIG. 2A is a partial side view of the distal end of the elongate shaft of the example medical device of FIG. 2 in an expanded filtering configuration;

FIG. 3 is a partial side view of an example medical device in an expanded filtering configuration;

FIG. 4 is a partial side view of an example medical device in an expanded filtering configuration;

FIG. 5 is a partial side view of an example medical device in an expanded filtering configuration;

FIG. 6 is a partial schematic representation of patient's aortic arch and aortic valve; and

FIGS. 7-13 are partial schematic representations of an example medical device being used within a patient's aortic arch and aortic valve in accordance with a method of use of the example medical device.

While the invention is amenable to various modifications and alternative forms, specifics thereof have been shown by way of example in the drawings and will be described in greater detail below. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

The terms “upstream” and “downstream” refer to a position or location relative to the direction of blood flow through a particular element or location, such as a vessel (i.e., the aorta), a heart valve (i.e., the aortic valve), and the like.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout several views. The detailed description and drawings are intended to illustrate but not limit the claimed invention.

A human heart includes several different heart valves, including aortic, pulmonary, mitral, and tricuspid valves, which control the flow of blood to and from the heart. Over time, a heart valve may become obstructed, narrowed, and/or less flexible (i.e., stenosed) due to hardening, calcium deposition, or other factors, thereby reducing the flow of blood through the valve and/or increasing the pressure within the chambers of the heart as the heart attempts to pump the blood through the vasculature. In some cases, aortic valve stenosis may result in the valve leaflets becoming fused together by calcium deposits, such as, for example, on the aortic or downstream side of the valve. One traditional treatment method is valve replacement, where the stenosed valve is removed and a replacement tissue or mechanical valve is implanted via open heart surgery. For some patients, an alternative to valve replacement may be valve repair, where the native heart valve is repaired percutaneously, to improve the function and/or extend the useful life of the heart valve without subjecting the patient to the invasiveness of open heart surgery. Percutaneous valve repair may include, for example, balloon valvuloplasty, and/or valvectomy and transcatheter aortic valve implantation, among other methods.

The devices and methods described herein are discussed with preference toward treatment of the aortic heart valve. However, it is fully contemplated that the devices and methods described herein may be adapted for use in the treatment of a non-aortic heart valve. One of ordinary skill in the art will understand that in the event of treating a non-aortic heart valve, the relative orientations and directions associated with the described devices and methods may be modified to accommodate the specifics (i.e., orientation, location, size, etc.) of the heart valve undergoing treatment.

In some embodiments, a percutaneously-deployable medical device may be employed to repair a heart valve. A medical device may be introduced into the vasculature and advanced through the aorta in a retrograde direction and into the aortic valve in a collapsed delivery configuration, with or without the aid of a separate delivery catheter. The medical device is then deployed to an expanded filtering configuration, where one or more of several functions or events may occur. The medical device may pre-dilitate the aortic valve for a subsequent procedure. The medical device may also, or alternatively, deploy a distal protection filter for use in a subsequent procedure, including, for example, the aforementioned pre-dilitation. In some embodiments, the medical device may deploy a distal protection filter before, or simultaneously to, pre-dilitating the aortic valve.

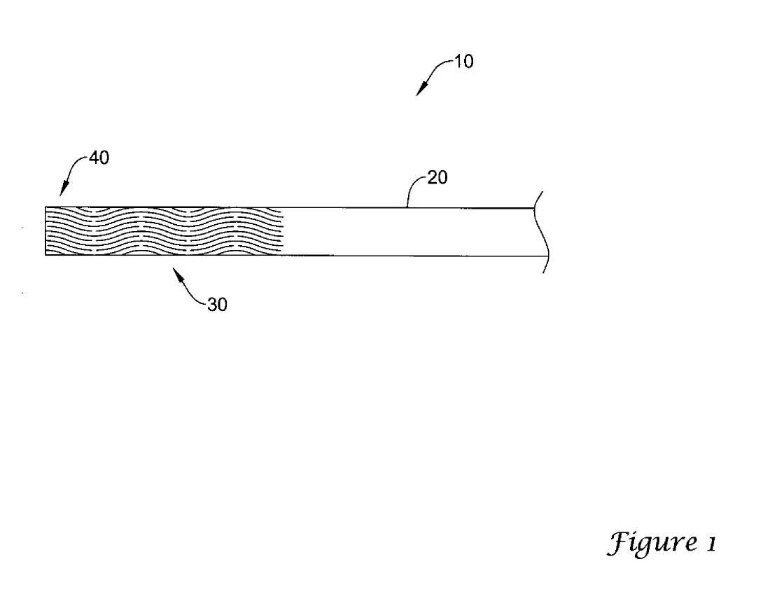

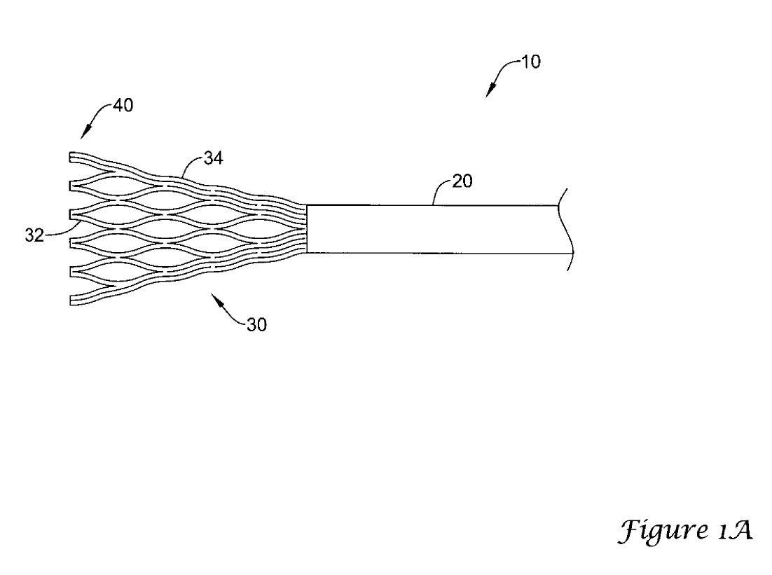

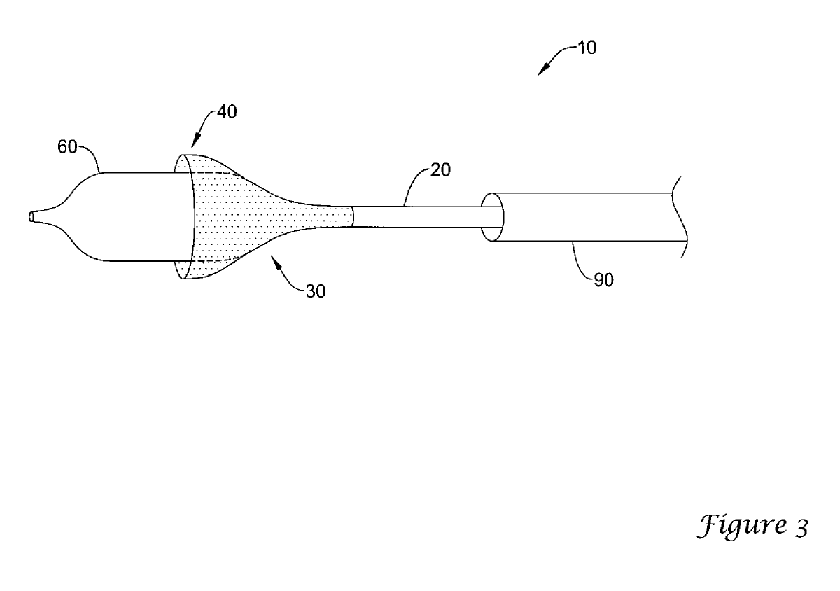

FIG. 1 illustrates a portion of an example medical device 10, such as those shown in FIGS. 3-5, which will be described in more detail herein. An example medical device 10 may comprise an elongate shaft 20 having a proximal end (not shown) and a distal end portion 30 including a distal end 40. As illustrated in FIGS. 1 and 1A, an example medical device 10 may include a distal end portion 30 configured to selectively expand from a collapsed delivery configuration to a distally-opening expanded filtering configuration. In some embodiments, the distal end 40 may have a first outer diameter in the collapsed delivery configuration and a second outer diameter in the expanded filtering configuration. In some embodiments, the distal end portion 30 may form a distal protection filter in the expanded filtering configuration.

In some embodiments, the distal end portion 30 may include a bistable structure. In some embodiments, a bistable structure may include a circumferentially-oriented support hoop 32 disposed at the distal end 40 of the elongate shaft 20. In some embodiments, a support hoop 32 may form a distal filter mouth. In some embodiments, a support hoop 32 may be disposed proximal of the distal end 40. In some embodiments, the support hoop 32 may be formed from the elongate shaft 20. In some embodiments, a bistable structure may include an expandable mesh 34 forming a plurality of apertures therethrough. The plurality of apertures of the expandable mesh 34 may facilitate perfusion blood flow through the expandable mesh 34 while capturing material larger than the apertures. In some embodiments, the expandable mesh 34 may be formed from the elongate shaft 20. In some embodiments, the entire distal end portion 30 may be formed into an expandable mesh 34. In general, when in the expanded filtering configuration, the distal end portion 30 forms a distally-opening distal protection filter generally expanding radially from an outer diameter of the elongate shaft 20, which is generally equivalent to the first outer diameter, distally toward the distal end 40. In some embodiments, the distal end portion 30 achieves its greatest outer extent at the distal end 40 in the expanded filtering configuration.

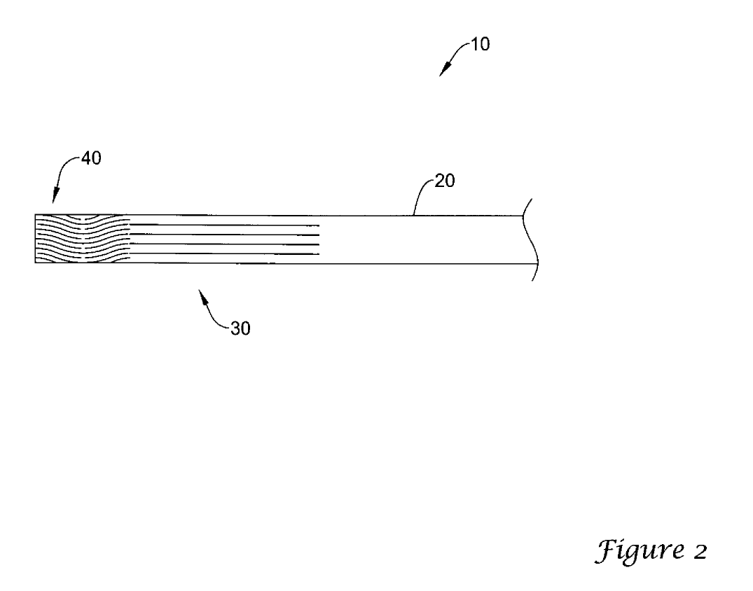

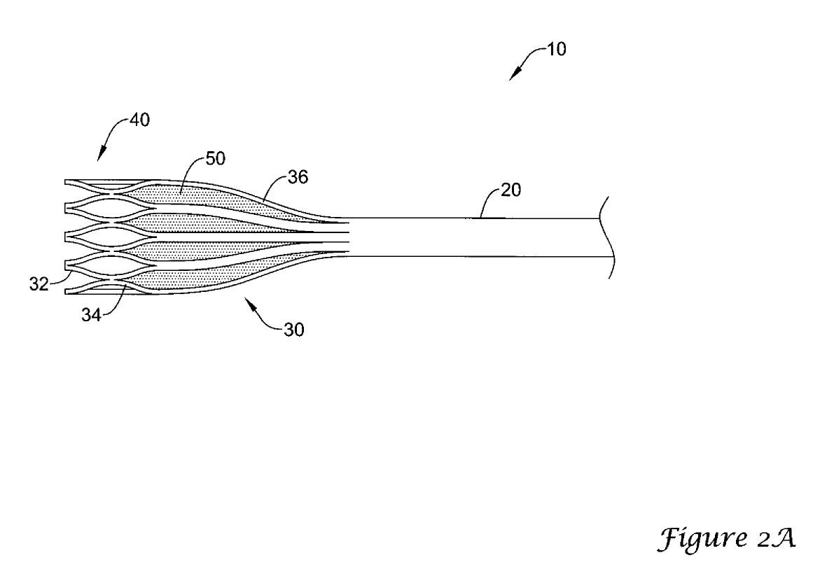

FIG. 2 illustrates a portion of an example medical device 10, such as those shown in FIGS. 3-5, which will be described in more detail herein. An example medical device 10 may comprise an elongate shaft 20 having a proximal end (not shown) and a distal end portion 30 including a distal end 40. As illustrated in FIGS. 2 and 2A, an example medical device 10 may include a distal end portion 30 configured to selectively expand from a collapsed delivery configuration to a distally-opening expanded filtering configuration. In some embodiments, the distal end 40 may have a first outer diameter in the collapsed delivery configuration and a second outer diameter in the expanded filtering configuration. In some embodiments, the distal end portion 30 may form a distal protection filter in the expanded filtering configuration.

In some embodiments, the distal end portion 30 may include a bistable structure. In some embodiments, a bistable structure may include a circumferentially-oriented support hoop 32 disposed at the distal end 40 of the elongate shaft 20. In some embodiments, a support hoop 32 may form a distal filter mouth. Although not expressly illustrated, in some embodiments, support hoop 32 may take other forms or shapes, such as a circular support hoop, an elliptical support hoop, or other suitable shapes. In some embodiments, a support hoop 32 may be disposed proximal of the distal end 40. In some embodiments, the support hoop 32 may be formed from the elongate shaft 20. In some embodiments, a bistable structure may include an expandable mesh 34. In some embodiments, the expandable mesh 34 may be formed from the elongate shaft 20. In some embodiments, a bistable structure may include a plurality of longitudinally-oriented struts 36 extending from a portion of the elongate shaft 20 proximal of the distal end portion 30 distally to the support hoop 32 and/or the distal end 40. In some embodiments, the plurality of struts 36 may be formed from the elongate shaft 20. In general, when in the expanded filtering configuration, the distal end portion 30 forms a distally-opening distal protection filter generally expanding radially from an outer diameter of the elongate shaft 20, which is generally equivalent to the first outer diameter, distally toward the distal end 40. In some embodiments, the distal end portion 30 achieves its greatest outer extent at the distal end 40 in the expanded filtering configuration. In some embodiments, a bistable structure may include a filter membrane 50 having a plurality of apertures therethrough disposed within the plurality of struts 36 and/or within an interior of the distal end portion 30. The plurality of apertures of the filter membrane 50 may facilitate perfusion blood flow through the filter membrane 50 while capturing material larger than the apertures. In some embodiments, a filter membrane 50 may be disposed within an expandable mesh 34 and/or a support hoop 32, if either of these features is present. The filter membrane 50, the distal end portion 30, and/or the elongate shaft 20 may include one or more coatings disposed thereon, such as an anti-thrombus coating, a hydrophilic coating, a hydrophobic coating, or other coatings suitable for the procedure being performed.

While not expressly illustrated, the distal end portion 30 of FIGS. 1A and 2A may further include a soft, flexible, and/or stretchable/expandable ring disposed on and/or about the distal end 40 of the elongate shaft 20. The ring may provide a sealing cushion against the wall of the aortic arch to reduce irritation or abrasion of the inner surface of the vessel wall as well as prevent the leakage of blood and/or embolic material around the distal protection filter. Additionally, a ring disposed on and/or about the distal end 40 may include a tether or actuation shaft (not shown) to close the ring and/or the distal end 40 prior to collapse and withdrawal of the distal end portion 30, so as to prevent captured embolic material from being released into the bloodstream during or after collapsing of the distal end portion 30.

The filter membrane 50 may be made of any suitable material, for example, a polymeric material, a thin-film metal or metal alloy, a metal-polymer composite, combinations thereof, and the like. Examples of suitable polymers may include polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials. Examples of suitable metallic materials may include stainless steels (e.g. 304v stainless steel), nickel-titanium alloys (e.g., nitinol, such as super elastic or linear elastic nitinol), nickel-chromium alloys, nickel-chromium-iron alloys, cobalt alloys, nickel, titanium, platinum, or other suitable materials, and the like.

The elongate shaft 20 and/or the distal end portion 30 may be made from materials such as metals, metal alloys, polymers, metal-polymer composites, or other suitable materials, and the like. In most embodiments, the elongate shaft 20 and the distal end portion 30 are made unitarily from the same material, although this is not required. Some examples of some suitable materials may include metallic materials and/or alloys such as stainless steel (e.g. 304v stainless steel or 316L stainless steel), nickel-titanium alloy (e.g., nitinol, such as super elastic or linear elastic nitinol), nickel-chromium alloy, nickel-chromium-iron alloy, cobalt alloy, nickel, titanium, platinum, or alternatively, a polymer material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some embodiments, portions of the medical device 10 may be made of, may be doped with, may include a layer of, or otherwise may include a radiopaque material. Radiopaque materials are understood to be materials capable of producing a relatively bright image on a fluoroscopy screen or another imaging technique such as X-ray during a medical procedure. This relatively bright image aids the user of device in determining its location. Suitable materials can include, but are not limited to, bismuth subcarbonate, iodine, gold, platinum, palladium, tantalum, tungsten or tungsten alloy, and the like.

FIG. 3 illustrates an example medical device 10 in accordance with the present disclosure. An example medical device 10 may comprise an elongate shaft 20 having a proximal end (not shown), a distal end portion 30 including a distal end 40, and a balloon 60 disposed at least partially within the distal end portion 30. For illustrative purposes, the distal end portion 30 is shown in FIG. 3 schematically, and may take the form shown in FIGS. 1-2A, or another suitable form as applicable. In some embodiments, the distal end portion 30 may be configured to selectively expand from a collapsed delivery configuration to a distally-opening expanded filtering configuration, in which the distal end portion 30 forms a distal protection filter having a mouth or major opening facing distally. In some embodiments, a medical device 10 may include a sheath 90 disposed about the elongate shaft 20 and sized to contain the distal end portion 30 and the balloon 60 within a lumen thereof. In some embodiments, the sheath 90 may include or be used as a delivery sheath and/or a retrieval sheath. In some embodiments, the sheath 90 may take the form of a catheter, sheath, hypotube, endoscope, or other tubular medical device suitable for the intended use.

As seen in FIG. 3, the balloon 60 may include a generally conical proximal waist, a generally conical distal waist, and a generally uniform diameter body portion disposed between the proximal waist and the distal waist. In some embodiments, the distal end portion 30 may be configured to selectively expand in response to inflation of the balloon 60. In operation, the balloon 60 may be in direct physical contact with the distal end portion 30 and/or the distal end 40 in the collapsed delivery configuration. In the collapsed delivery configuration, the distal end 40 may have a first outer diameter that is generally equal to an outer diameter of the elongate shaft 20, and the distal end 40 may be biased or self-biased radially inwardly. As the balloon 60 is inflated, the balloon 60 expands the distal end portion 30 and/or the distal end 40 radially outwardly toward a second outer diameter that is greater than the first outer diameter. In some embodiments, the balloon 60 is configured to at least partially expand the distal end portion 30 and/or at least partially expand the distal end 40 radially outwardly to at least an intermediate outer diameter. At an outer diameter less than the intermediate outer diameter, the distal end 40 may be biased or self-biased radially inwardly toward the first outer diameter. At an outer diameter larger than the intermediate outer diameter, the distal end 40 may be biased or self-biased radially outwardly toward the second outer diameter. In other words, the intermediate outer diameter may form or represent an equilibrium or transition point at which the distal end portion 30 and/or the distal end 40 springs or snaps either towards the collapsed delivery configuration or the expanded filtering configuration. For example, during inflation of the balloon 60, once the distal end 40 expands radially outwardly past the intermediate outer diameter, the distal end 40 will spring or snap to the second outer diameter, regardless of the inflation percentage of the balloon 60. That is, in some embodiments, the balloon 60 does not need to be fully inflated to fully expand the distal end portion 30 and/or the distal end 40 to the expanded filtering configuration. In the expanded filtering configuration, the distal end 40 of the elongate shaft 20 may extend to a greater outermost extent than the balloon 60. In other words, an inner diameter of the elongate shaft 20 at the distal end 40 may be greater than an outermost extent of the balloon 60, such that an inner surface of the elongate shaft 20 at the distal end 40 may be radially spaced apart from an outer surface of the balloon 60, in the expanded filtering configuration.

In some embodiments, the balloon 60 may be configured for placement at least partially within the aortic valve such that the distal end 40 of the elongate shaft 20 is disposed downstream of the aortic valve. For example, the body portion of the balloon 60 may be disposed within the aortic valve, with the distal waist disposed within the left ventricle and the proximal waist disposed within the aortic arch, although the exact placement may vary as needed or desired. In some embodiments, the balloon 60 may include a lumen passing longitudinally therethrough for the passage of a guidewire or other device. An inflation lumen (not shown) fluidly connected to the interior of the balloon 60 may be disposed within the elongate shaft 20, and may be fluidly connected to a port or manifold (not shown) at the proximal end of the elongate shaft 20.

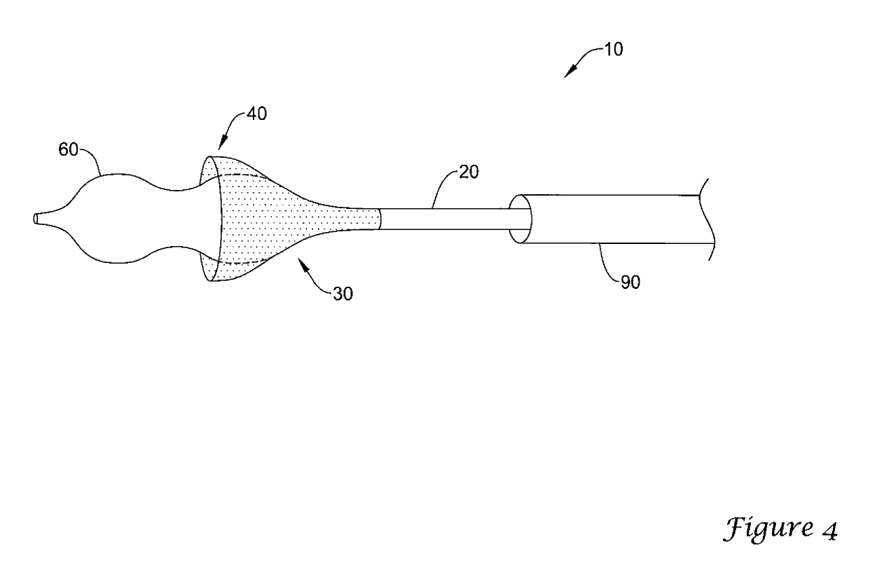

FIG. 4 illustrates an example medical device 10 in accordance with the present disclosure. An example medical device 10 may comprise an elongate shaft 20 having a proximal end (not shown), a distal end portion 30 including a distal end 40, and an hourglass-shaped balloon 60 disposed at least partially within the distal end portion 30. For illustrative purposes, the distal end portion 30 is shown in FIG. 4 schematically, and may take the form shown in FIGS. 1-2A, or another suitable form as applicable. In some embodiments, the distal end portion 30 may be configured to selectively expand from a collapsed delivery configuration to a distally-opening expanded filtering configuration, in which the distal end portion 30 forms a distal protection filter having a mouth or major opening facing distally. In some embodiments, a medical device 10 may include a sheath 90 disposed about the elongate shaft 20 and sized to contain the distal end portion 30 and the hourglass-shaped balloon 60 within a lumen thereof. In some embodiments, the sheath 90 may include or be used as a delivery sheath and/or a retrieval sheath. In some embodiments, the sheath 90 may take the form of a catheter, sheath, hypotube, endoscope, or other tubular medical device suitable for the intended use.

As seen in FIG. 4, the hourglass-shaped balloon 60 may include a generally conical proximal waist, a generally conical distal waist, and an hourglass-shaped body portion (i.e. a narrowed central portion disposed axially between widened proximal and distal portions) disposed between the proximal waist and the distal waist. In some embodiments, the distal end portion 30 may be configured to selectively expand in response to inflation of the hourglass-shaped balloon 60. In operation, the hourglass-shaped balloon 60 may function similar to the balloon 60 of FIG. 3. For example, the hourglass-shaped balloon 60 may be in direct physical contact with the distal end portion 30 and/or the distal end 40 in the collapsed delivery configuration. In the collapsed delivery configuration, the distal end 40 may have a first outer diameter that is generally equal to an outer diameter of the elongate shaft 20, and the distal end 40 may be biased or self-biased radially inwardly. As the hourglass-shaped balloon 60 is inflated, the hourglass-shaped balloon 60 expands the distal end portion 30 and/or the distal end 40 radially outwardly toward a second outer diameter that is greater than the first outer diameter. In some embodiments, the hourglass-shaped balloon 60 is configured to at least partially expand the distal end portion 30 and/or at least partially expand the distal end 40 radially outwardly to at least an intermediate outer diameter. At an outer diameter less than the intermediate outer diameter, the distal end 40 may be biased or self-biased radially inwardly toward the first outer diameter. At an outer diameter larger than the intermediate outer diameter, the distal end 40 may be biased or self-biased radially outwardly toward the second outer diameter. In other words, the intermediate outer diameter may form or represent an equilibrium or transition point at which the distal end portion 30 and/or the distal end 40 springs or snaps either towards the collapsed delivery configuration or the expanded filtering configuration. For example, during inflation of the balloon 60, once the distal end 40 expands radially outwardly past the intermediate outer diameter, the distal end 40 will spring or snap to the second outer diameter, regardless of the inflation percentage of the balloon 60. That is, in some embodiments, the balloon 60 does not need to be fully inflated to fully expand the distal end portion 30 and/or the distal end 40 to the expanded filtering configuration. In the expanded filtering configuration, the distal end 40 of the elongate shaft 20 may extend to a greater outermost extent than the hourglass-shaped balloon 60. In other words, an inner diameter of the elongate shaft 20 at the distal end 40 may be greater than an outermost extent of the hourglass-shaped balloon 60, such that an inner surface of the elongate shaft 20 at the distal end 40 may be radially spaced apart from an outer surface of the hourglass-shaped balloon 60, in the expanded filtering configuration.

In some embodiments, the hourglass-shaped balloon 60 may be configured for placement at least partially within the aortic valve such that the distal end 40 of the elongate shaft 20 is disposed downstream of the aortic valve. For example, the hourglass-shaped body portion of the hourglass-shaped balloon 60 may be disposed within the aortic valve (i.e. the narrowed central portion may be positioned within the aortic valve), with the distal waist disposed within the left ventricle and the proximal waist disposed within the aortic arch, although the exact placement may vary as needed or desired. In some embodiments, the hourglass-shaped balloon 60 may include a lumen passing longitudinally therethrough for the passage of a guidewire or other device. An inflation lumen (not shown) fluidly connected to the interior of the hourglass-shaped balloon 60 may be disposed within the elongate shaft 20, and may be fluidly connected to a port or manifold (not shown) at the proximal end of the elongate shaft 20.

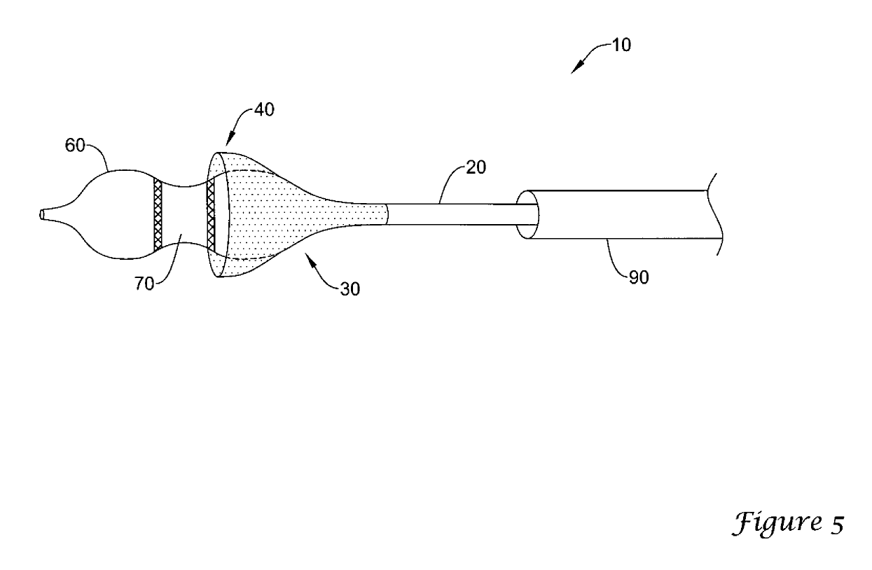

FIG. 5 illustrates an example medical device 10 in accordance with the present disclosure. An example medical device 10 may comprise an elongate shaft 20 having a proximal end (not shown), a distal end portion 30 including a distal end 40, and an hourglass-shaped balloon 60 disposed at least partially within the distal end portion 30.

For illustrative purposes, the distal end portion 30 is shown in FIG. 5 schematically, and may take the form shown in FIGS. 1-2A, or another suitable form as applicable. Substantially similar to the example medical device 10 of FIG. 4 in form and function, the example medical device 10 of FIG. 5 may further include a transcatheter aortic valve implantation device 70 disposed on the hourglass-shaped balloon 60, for example, on a narrowed central portion. Similar to the example medical device 10 of FIG. 4, in some embodiments, the distal end portion 30 may be configured to selectively expand from a collapsed delivery configuration to a distally-opening expanded filtering configuration, in which the distal end portion 30 forms a distal protection filter having a mouth or major opening facing distally. In some embodiments, a medical device 10 may include a sheath 90 disposed about the elongate shaft 20 and sized to contain the distal end portion 30 and the hourglass-shaped balloon 60 within a lumen thereof. In some embodiments, the sheath 90 may include or be used as a delivery sheath and/or a retrieval sheath. In some embodiments, the sheath 90 may take the form of a catheter, sheath, hypotube, endoscope, or other tubular medical device suitable for the intended use.

As seen in FIG. 5, the hourglass-shaped balloon 60 may include a generally conical proximal waist, a generally conical distal waist, and an hourglass-shaped body portion (i.e. a narrowed central portion disposed axially between widened proximal and distal portions) disposed between the proximal waist and the distal waist. In some embodiments, the distal end portion 30 may be configured to selectively expand in response to inflation of the hourglass-shaped balloon 60. In operation, the hourglass-shaped balloon 60 of FIG. 5 may function similar to the balloon 60 of FIGS. 3 and 4. For example, the hourglass-shaped balloon 60 may be in direct physical contact with the distal end portion 30 and/or the distal end 40 in the collapsed delivery configuration. In the collapsed delivery configuration, the distal end 40 may have a first outer diameter that is generally equal to an outer diameter of the elongate shaft 20, and the distal end 40 may be biased or self-biased radially inwardly. As the hourglass-shaped balloon 60 is inflated, the hourglass-shaped balloon 60 expands the distal end portion 30 and/or the distal end 40 radially outwardly toward a second outer diameter that is greater than the first outer diameter. In some embodiments, the hourglass-shaped balloon 60 is configured to at least partially expand the distal end portion 30 and/or at least partially expand the distal end 40 radially outwardly to at least an intermediate outer diameter. At an outer diameter less than the intermediate outer diameter, the distal end 40 may be biased or self-biased radially inwardly toward the first outer diameter. At an outer diameter larger than the intermediate outer diameter, the distal end 40 may be biased or self-biased radially outwardly toward the second outer diameter. In other words, the intermediate outer diameter may form or represent an equilibrium or transition point at which the distal end portion 30 and/or the distal end 40 springs or snaps either towards the collapsed delivery configuration or the expanded filtering configuration. For example, during inflation of the balloon 60, once the distal end 40 expands radially outwardly past the intermediate outer diameter, the distal end 40 will spring or snap to the second outer diameter, regardless of the inflation percentage of the balloon 60. That is, in some embodiments, the balloon 60 does not need to be fully inflated to fully expand the distal end portion 30 and/or the distal end 40 to the expanded filtering configuration. In the expanded filtering configuration, the distal end 40 of the elongate shaft 20 may extend to a greater outermost extent than the hourglass-shaped balloon 60. In other words, an inner diameter of the elongate shaft 20 at the distal end 40 may be greater than an outermost extent of the hourglass-shaped balloon 60, such that an inner surface of the elongate shaft 20 at the distal end 40 may be radially spaced apart from an outer surface of the hourglass-shaped balloon 60, in the expanded filtering configuration.

In some embodiments, the hourglass-shaped balloon 60 may be configured for placement at least partially within the aortic valve such that the distal end 40 of the elongate shaft 20 is disposed downstream of the aortic valve. For example, the hourglass-shaped body portion of the hourglass-shaped balloon 60 may be disposed within the aortic valve (i.e. the narrowed central portion having a transcatheter aortic valve implantation device 70 disposed thereon may be positioned within the aortic valve), with the distal waist disposed within the left ventricle and the proximal waist disposed within the aortic arch, although the exact placement may vary as needed or desired. In some embodiments, the hourglass-shaped balloon 60 may include a lumen passing longitudinally therethrough for the passage of a guidewire or other device. An inflation lumen (not shown) fluidly connected to the interior of the hourglass-shaped balloon 60 may be disposed within the elongate shaft 20, and may be fluidly connected to a port or manifold (not shown) at the proximal end of the elongate shaft 20.

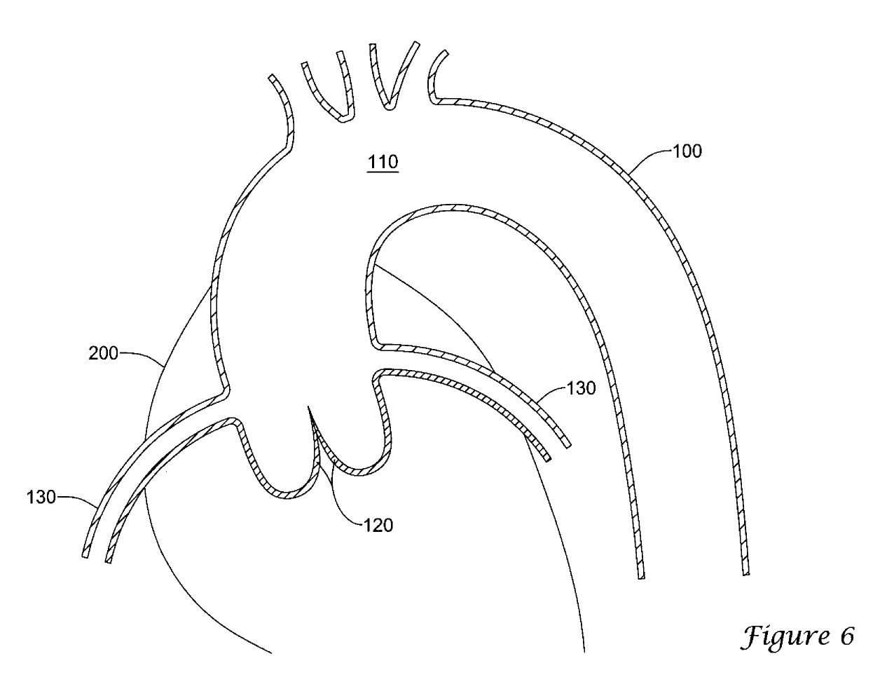

FIG. 6 shows a schematic representation of a portion of the cardiac system of a patient, including the heart 200, the aorta 100, the aortic arch 110, the aortic valve 120, and the coronary arteries 130. The aortic valve 120 may comprise a plurality of valve leaflets, typically two to four leaflets, opening distally into the aortic arch 110.

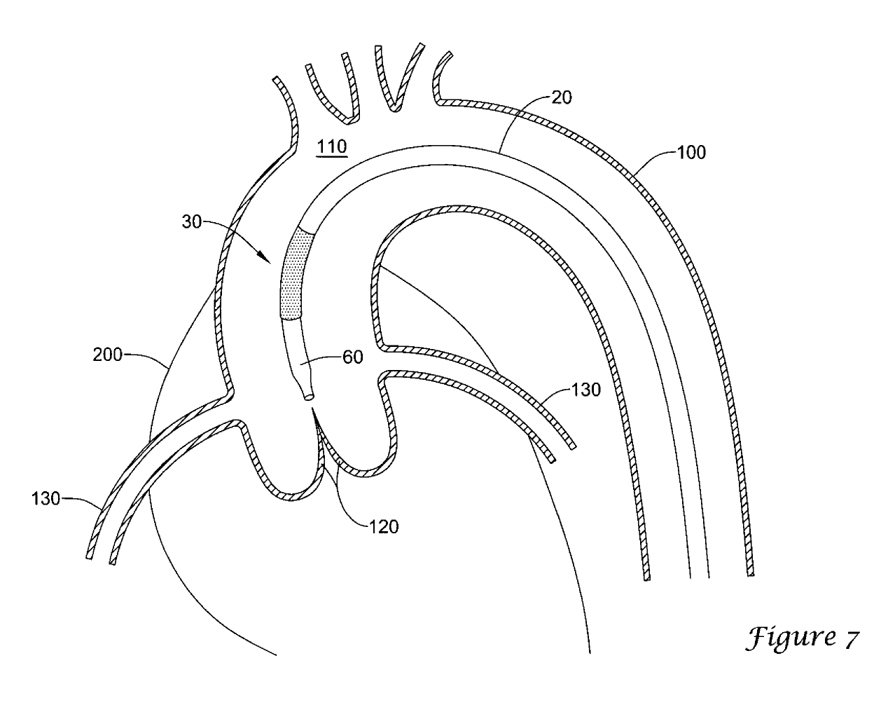

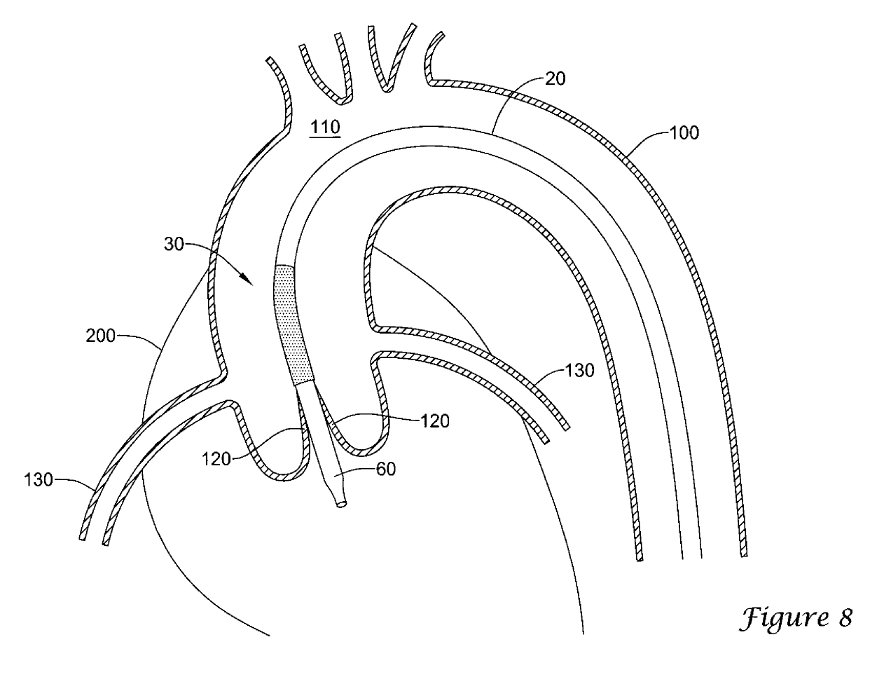

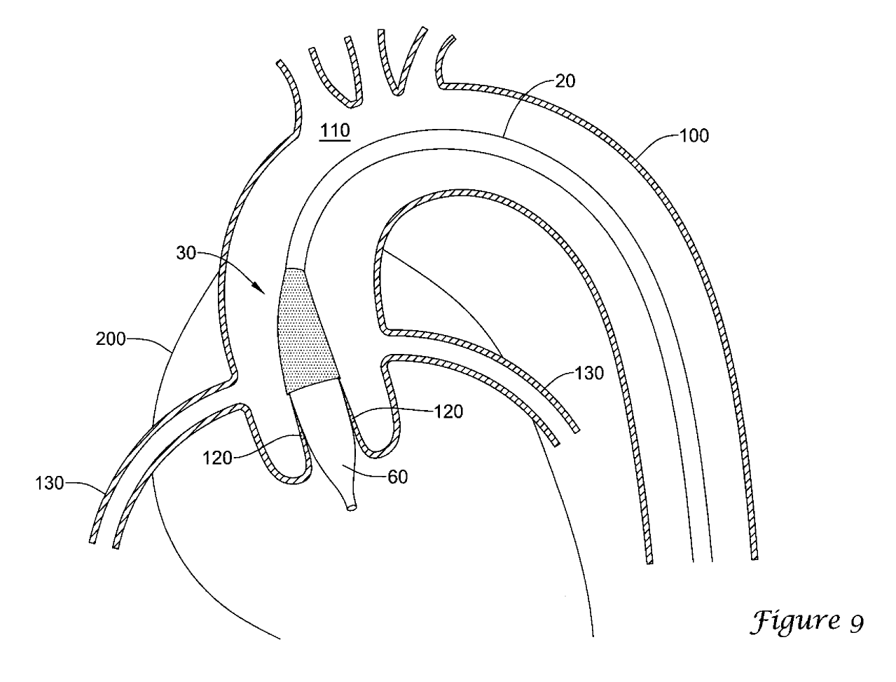

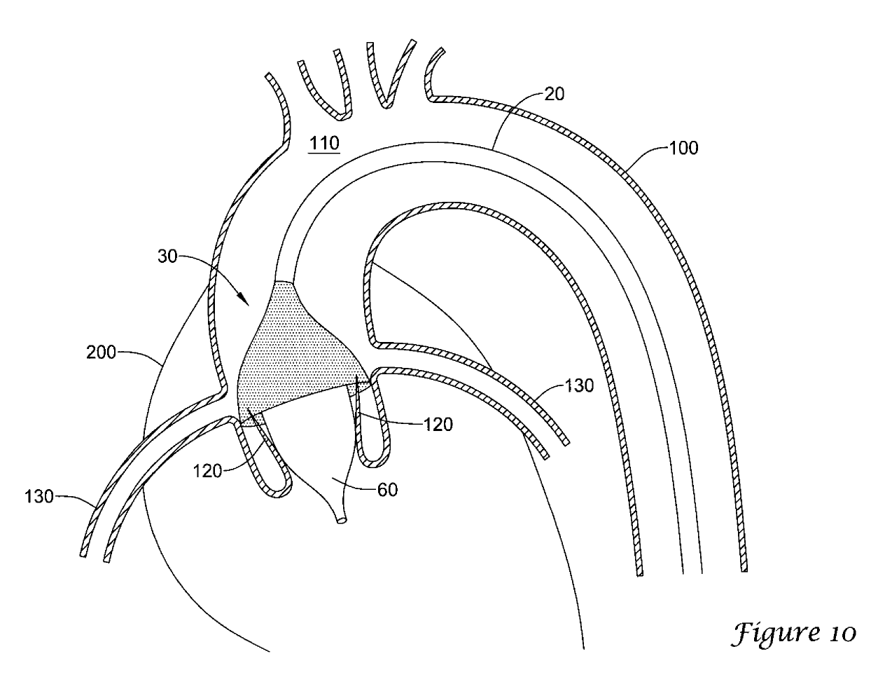

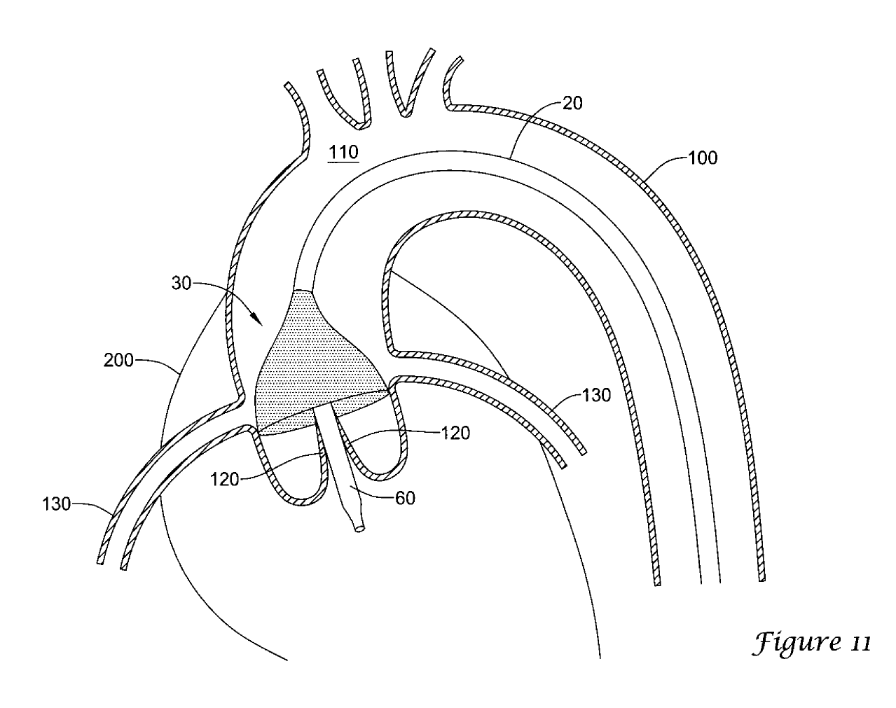

In operation, an example medical device 10 of FIGS. 3-5 may be advanced in a retrograde direction through the vasculature to a position adjacent to the treatment site (i.e., the aortic valve 120), as seen in FIG. 7. In some embodiments, the medical device 10 may be advanced with the aid of a pre-positioned guidewire (not shown) disposed within a lumen of the medical device 10. As seen in FIG. 8, the balloon 60, in the collapsed delivery configuration, may be extended through the treatment site (i.e., the aortic valve 120) and into the left ventricle such that a distalmost tip of the balloon 60 is disposed distal or upstream of the treatment site (i.e., the aortic valve 120) and the distal end 40 of the elongate shaft 20 is disposed adjacent to and/or proximal or downstream of the treatment site (i.e., the aortic valve 120), with a central body portion of the balloon 60 disposed generally within an opening of the treatment site (i.e., the aortic valve 120). In some embodiments, the distal end 40 of the elongate shaft 20 may be positioned distally or upstream of the coronary arteries 130 and/or the carotid arteries. Once the medical device 10 is positioned, the balloon 60 may be at least partially inflated, as seen in FIG. 9, toward the expanded filtering configuration. During initial inflation/expansion, the distal end portion 30 and/or the distal end 40 is biased or self-biased radially inwardly toward the first outer diameter and/or the collapsed delivery configuration. Upon inflation to at least the intermediate outer diameter, the distal end portion 30 and/or the distal end 40 may transition from an inwardly-biased state to an outwardly-biased state, and the distal end 40 may spring or snap radially outwardly to the second outer diameter and substantially conform to an inner surface of the vessel adjacent to the treatment site (i.e. the aortic arch 110). After the distal end portion 30 and/or the distal end 40 have been expanded radially outwardly to the expanded filtering configuration, the balloon 60 may be inflated to its fully expanded outer diameter, as seen in FIG. 10, where an outer surface of the balloon 60 is in contact with the treatment site (i.e. the valve leaflets of the aortic valve 120), thereby “cracking” or breaking open a stenosed or calcified aortic valve 120 and/or pre-dilitating the aortic valve 120. The distal end portion 30, in the expanded filtering configuration, forms a distal protection filter to capture embolic material dislodged by the balloon 60, while permitting perfusion blood flow through the aorta 100. Next, the balloon 60 may be deflated, as seen in FIG. 11, and another percutaneous medical procedure (i.e. a valvectomy, a valvuloplasty, or a transcatheter aortic valve implantation procedure) subsequently performed with the distal end portion 30 held in a constant position within the vessel adjacent to the treatment site (i.e. the aortic valve 120) in the expanded filtering configuration.

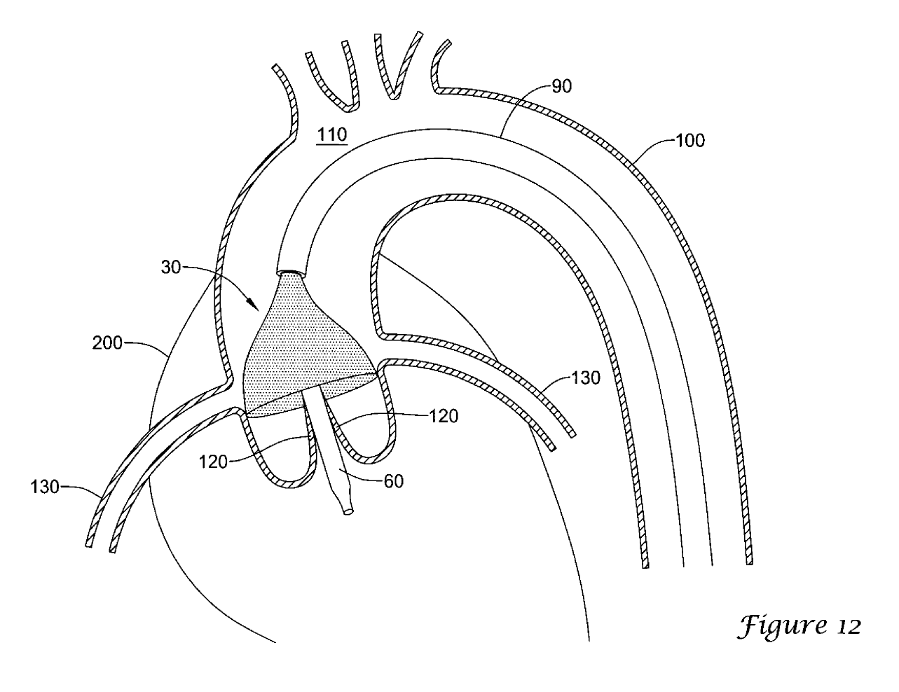

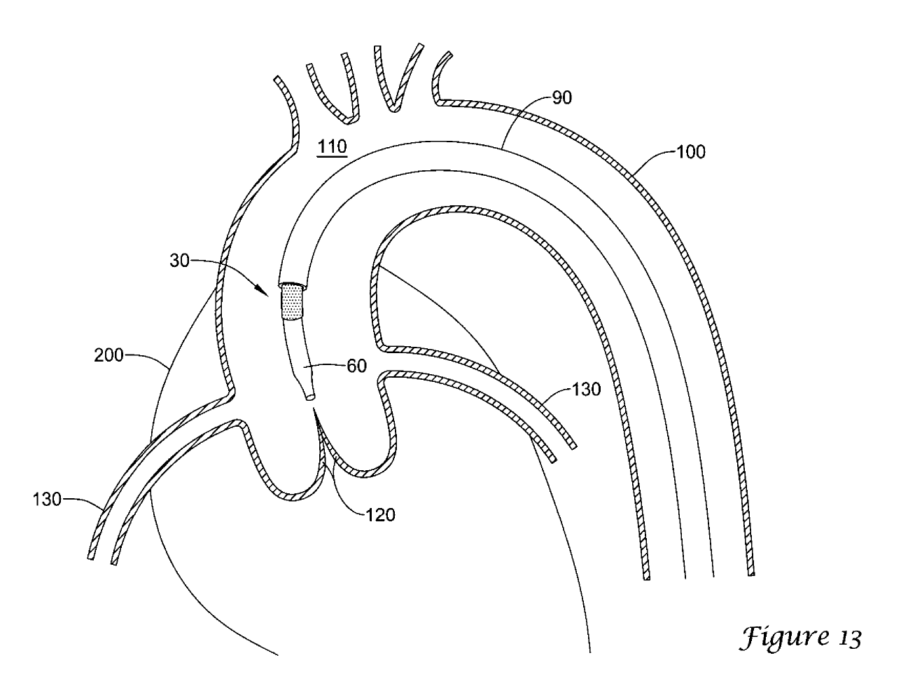

In some embodiments, the balloon 60 may be re-used in a subsequent procedure. In some embodiments, the balloon 60 may be withdrawn through the elongate shaft 20 prior to performing a subsequent procedure and the elongate shaft 20 may be used as a delivery sheath for another medical device to be used in the subsequent procedure. In some embodiments, after the balloon 60 has been collapsed and/or after a subsequent procedure (if a subsequent procedure occurs), a sheath 90 may be advanced into contact with the distal end portion 30, as seen in FIG. 12, and the distal end portion 30 and/or the distal end 40 may be collapsed into the collapsed delivery configuration for withdrawal from the treatment site, as seen in FIG. 13.

Although not expressly illustrated, a portion of the sheath 90 and/or the elongate shaft 20 proximal to distal end portion 30 may be configured to include a predetermined bending configuration aligning with the curve of the aorta 100 and/or the aortic arch 110. For example, in some embodiments, the elongate shaft 20 may include a directional bending component (not shown) that aligns the elongate shaft 20 and/or the sheath 90 with the curve of the aorta 100 and/or the aortic arch 110. For example, the elongate shaft 20 may include a metallic wire or strip (not shown) embedded within a wall of the elongate shaft 20 or disposed within a lumen within the wall of the elongate shaft 20. The metallic wire or strip may be flattened or otherwise configured to have a predetermined or preferential bending direction. As the elongate shaft 20 is advanced through the aortic arch 110, the elongate shaft 20 may align such that the balloon 60 will assume a predetermined orientation within the treatment site (i.e., the aortic valve 120). In some embodiments, the predetermined orientation may correspond to one or more of the openings between the valve leaflets of the aortic valve 120.

It should be understood that although the above discussion was focused on a medical device and methods of use within the coronary vascular system of a patient, other embodiments of medical devices or methods in accordance with the invention can be adapted and configured for use in other parts of the anatomy of a patient. For example, devices and methods in accordance with the invention can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the medical devices described herein with respect to percutaneous deployment may be used in other types of surgical procedures as appropriate. For example, in some embodiments, the medical devices may be deployed in a non-percutaneous procedure, including an open heart procedure. Devices and methods in accordance with the invention can also be adapted and configured for other uses within the anatomy.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

We claim:

1. A medical device comprising:
an elongate shaft having a proximal end, a distal end portion including a distal end, and a balloon disposed at least partially within the distal end portion;
wherein the distal end portion is configured to selectively expand from a collapsed delivery configuration to a distally-opening expanded filtering configuration;
wherein the distal end has a first outer diameter in the collapsed delivery configuration and a second outer diameter in the expanded filtering configuration.

2. The medical device of claim 1, wherein the distal end portion is configured to selectively expand in response to inflation of the balloon.

3. The medical device of claim 2, wherein the balloon expands the distal end to the second outer diameter.

4. The medical device of claim 2, wherein the balloon is configured to at least partially expand the distal end to at least an intermediate outer diameter, wherein at an outer diameter larger than the intermediate outer diameter, the distal end is self-biased toward the second outer diameter.

5. The medical device of claim 4, wherein the distal end portion includes a bistable structure.

6. The medical device of claim 5, wherein at an outer diameter less than the intermediate outer diameter, the distal end is self-biased toward the first outer diameter.

7. The medical device of claim 5, wherein the bistable structure includes a circumferentially-oriented support hoop formed from the elongate shaft.

8. The medical device of claim 5, wherein the bistable structure includes an expandable mesh formed from the elongate shaft.

9. The medical device of claim 5, wherein the bistable structure includes a plurality of longitudinally-oriented struts formed from the elongate shaft.

10. The medical device of claim 9, wherein the bistable structure includes a filter membrane disposed within the plurality of longitudinally-oriented struts.

11. The medical device of claim 1, wherein the distal end portion forms a distal protection filter in the expanded filtering configuration.

12. The medical device of claim 11, wherein an inner diameter of the elongate shaft at the distal end in the expanded filtering configuration is greater than an outermost extent of the balloon.

13. The medical device of claim 12, wherein the distal end of the elongate shaft is radially spaced apart from the balloon in the expanded filtering configuration.

14. The medical device of claim 1, wherein the distal end portion is formed from a shape memory material configured to self-expand to the expanded filtering configuration.

15. The medical device of claim 1, wherein the balloon is configured for placement at least partially within an aortic valve such that the distal end of the elongate shaft is disposed downstream of the aortic valve.

16. The medical device of claim 1, wherein the medical device further includes a transcatheter aortic valve implantation device disposed on the balloon.

17. A method of deploying a medical device at a treatment site, comprising:
obtaining an elongate shaft having a proximal end, a distal end portion including a distal end, and a balloon disposed at least partially within the distal end portion and extending distally therefrom, wherein the distal end portion is configured to selectively expand from a collapsed delivery configuration to a distally-opening expanded filtering configuration;
advancing the elongate shaft through a vessel in a retrograde direction to the treatment site;
disposing at least a portion of the balloon within the treatment site such that the distal end of the elongate shaft is positioned adjacent the treatment site;
at least partially inflating the balloon, thereby expanding the distal end of the elongate shaft to the expanded filtering configuration, wherein the distal end substantially conforms to an inner surface of the vessel adjacent to the treatment site; and
deflating the balloon and subsequently performing a valvectomy, valvuloplasty, or transcatheter aortic valve implantation procedure with the distal end of the elongate shaft positioned within the vessel adjacent to the treatment site in the expanded filtering configuration.

18. The method of claim 17, wherein at least partially inflating the balloon transitions the distal end portion from an inwardly-biased state to an outwardly-biased state.

19. The method of claim 18, wherein the distal end portion includes a bistable structure.

20. The method of claim 17, wherein in the expanded filtering configuration, the distal end of the elongate shaft is radially spaced apart from the balloon.
